(19) United States
(12) Patent Application Publication
(10) Pub. No.: US 2025/0352143 A1
(43) Pub. Date: Nov. 20, 2025

(54) METHOD AND SYSTEM FOR DEFINING STRESS LEVEL

(71) Applicant: Summa Finland Oy, Oulu (FI)

(72) Inventors: Aleksi Juurikka, Oulu (FI); Juuso Nissilä, Oulu (FI); Tommi Kinnunen, Helsinki (FI); Marko Mäkinen, Oulu (FI); Wojciech Kurek, Wroclaw (PL)

(73) Assignee: Summa Finland Oy, Oulu (FI)

(21) Appl. No.: 19/206,594

(22) Filed: May 13, 2025

(30) Foreign Application Priority Data
May 14, 2024 (FI) 20245606

(51) Int. Cl. A61B 5/00; G16H 10/20

(52) U.S. Cl. CPC A61B 5/4884; G16H 10/20

(57) ABSTRACT

A method for defining a stress level, the method including obtaining a first set of information related to mental stress from a user via a first questionary; obtaining a second set of information related to physical stress from the user via a second questionary; measuring a hormonal level at a first moment of time; using the first and second sets of information and the measured hormonal level to derive a stress reaction over a pre-defined period of time; validating the stress reaction using longitudinal stress reaction; and defining the stress level by modelling stress level from at least one of: the stress reaction from at least one of: the first and second questionaries; the validated stress reaction.

### TECHNICAL FIELD

The present disclosure relates to methods for defining stress levels. Moreover, the present disclosure relates to systems for defining stress levels.

### BACKGROUND

Generally, stress plays a major role in human health and well-being, with both mental and physical stressors contributing to a wide range of adverse outcomes. Chronic stress has been linked to serious health issues such as cardiovascular diseases, mental health disorders, weakened immune responses and the like. Despite its significant impact, accurately measuring and analysing the stress remains a complex challenge due to its multifaceted nature, involving mental, physical, and physiological components. The ability to comprehensively assess stress levels and their consequences is essential for improving health outcomes, enhancing performance, and promoting overall well-being.

Existing solutions for measuring the stress primarily rely on isolated approaches, such as wearable devices that monitor heart rate variability (HRV) or questionnaires that assess perceived mental stress. While the aforementioned methods provide some insights, they are often limited in scope. For example, HRV-based devices can measure acute stress but fail to distinguish between beneficial and harmful stressors or provide insights into recovery. Similarly, mental stress questionnaires are subjective and prone to bias, as they rely on self-reported data that may not accurately reflect the user's actual stress state. The aforementioned methods, while useful in specific contexts, lack the ability to provide a holistic view of the stress that integrates mental, physical, and physiological dimensions.

Furthermore, hormonal stress analysis, which involves measuring biomarkers, has been explored in prior art as a means to assess stress. However, these methods are often limited to single-point measurements, such as morning or evening samples, and fail to account for the dynamic nature of stress throughout the day. Additionally, existing hormonal analysis methods do not adequately combine stress data, resulting in an incomplete understanding of stress reactions and recovery processes.

Therefore, in light of the foregoing discussion, there exists a need to overcome the aforementioned drawbacks.

### SUMMARY

The aim of the present disclosure is to provide a method and a system to accurately define and analyse an individual's stress level by obtaining information related to mental stress and physical stress from a user and by measuring at least one hormonal level of the individual. The aim of the present disclosure is achieved by a method and a system for defining a stress level as defined in the appended independent claims to which reference is made to. Advantageous features are set out in the appended dependent claims.

Throughout the description and claims of this specification, the words “comprise”, “include”, “have”, and “contain” and variations of these words, for example “comprising” and “comprises”, mean “including but not limited to”, and do not exclude other components, items, integers or steps not explicitly disclosed also to be present. Moreover, the singular encompasses the plural unless the context otherwise requires. In particular, where the indefinite article is used, the specification is to be understood as contemplating plurality as well as singularity, unless the context requires otherwise.

### BRIEF DESCRIPTION OF THE DRAWINGS

FIG. 1 is an illustration of a flow chart depicting steps of a method for defining a stress level, in accordance with an embodiment of the present disclosure;

FIG. 2 is an illustration of a block diagram of a system for defining a stress level, in accordance with an embodiment of the present disclosure;

FIG. 3 is an illustration of an exemplary implementation of a stress level analysis, in accordance with an embodiment of the present disclosure;

FIG. 4 is an illustration of a graphical representation of a stress level analysis, in accordance with an embodiment of the present disclosure;

FIG. 5 is a schematic illustration of a graph for assessing the duration and magnitude of a stress reaction, in accordance with an embodiment of the present disclosure;

FIG. 6 is an illustration of a graphical representation of a population sample for real-time, single-point hormonal stress analysis, in accordance with an embodiment of the present disclosure;

FIG. 7 is an illustration of an exemplary implementation of a set of predetermined tags, in accordance with an embodiment of the present disclosure;

FIG. 8 is an illustration of exemplary behaviour of a transformation function of a set of predetermined tags, in accordance with an embodiment of the present disclosure;

FIG. 9 is an illustration of a graphical representation depicting the distribution of user's adjusted mental load and flow score, in accordance with an embodiment of the present disclosure;

FIG. 10 is an illustration of a graphical representation depicting balancing of the user's adjusted mental load and flow score, in accordance with an embodiment of the present disclosure;

FIG. 11 is an illustration of a table presenting the questions to assess exertion of a strength training session, in accordance with an embodiment of the present disclosure;

FIG. 12 is an illustration of a graphical representation depicting modelling of the intensity (exertion) distribution, in accordance with an embodiment of the present disclosure;

FIG. 13 is an illustration of a graphical representation depicting heart rate zone distribution modeling during a training session of a user, in accordance with an embodiment of the present disclosure;

FIG. 14 is an illustration of a boxplot representation depicting stress distribution across different heart rate training zones during exercise sessions of a user, in accordance with an embodiment of the present disclosure;

FIG. 15 is an illustration of a boxplot representation depicting statistical distribution of training session time percentages in different heart rate zones, in accordance with an embodiment of the present disclosure;

FIG. 16 is an illustration of a boxplot representation depicting statistical distribution of training session time percentages in different heart rate zones, in accordance with another embodiment of the present disclosure;

FIG. 17 is an illustration of a tabular representation depicting analysis of intensity distribution across two consecutive days, in accordance with an embodiment of the present disclosure;

FIG. 18 is an illustration of a graphical representation depicting stress and recovery of a user in a user interface, in accordance with an embodiment of the present disclosure;

FIG. 19 is an illustration of a graphical representation depicting a stress modelling approach of an exemplary scenario, in accordance with an embodiment of the present disclosure;

FIG. 20 is an illustration of a flow chart depicting steps of a stress evaluation method for physical stress, in accordance with an embodiment of the present disclosure;

FIG. 21 is an illustration of a graphical representation depicting fatigue and stress accumulation during a strength training session, in accordance with an embodiment of the present disclosure;

FIG. 22 is an illustration of a graphical representation depicting fatigue and stress accumulation during a single exercise set, in accordance with an embodiment of the present disclosure; and

FIG. 23 is an illustration of a graphical representation depicting fatigue and stress accumulation across multiple sets and repetitions, in accordance with an embodiment of the present disclosure.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practising the present disclosure are also possible.

In a first aspect, the present disclosure provides a method for defining a stress level, the method comprising:
obtaining a first set of information related to mental stress from a user via a first questionary;
obtaining a second set of information related to physical stress from the user via a second questionary;
measuring a hormonal level at a first moment of time;
using the first and second sets of information and the measured hormonal level to derive a stress reaction over a pre-defined period of time;
validating the stress reaction using longitudinal stress reaction; and
defining the stress level by modelling stress level from at least one of:
   the stress reaction from at least one of: the first and second questionaries;
   the validated stress reaction.

The present disclosure provides the aforementioned method to facilitate an accurate, efficient detection and monitoring of an individual's stress level by integrating subjective and objective data sources. It provides actionable insights about the user's stress level that support personal health management, clinical decision-making, and wellness optimization. The method combines self-reported mental and physical stress with real-time hormonal measurements, thereby enabling a more comprehensive and validated assessment of stress and recovery dynamics. Moreover, acquisition of the first set of information and the second set of information ensures that the method captures full spectrum of stressors affecting the individual, including both psychological and physiological domains. The use of targeted questionaries allows for the efficient and user-friendly collection of relevant data, tailored to the individual's daily experiences and activities. Furthermore, the measurement of the hormonal level introduces an objective, biological marker into the assessment, providing a real-time picture of the body's physiological stress response. The integration of the subjective and the objective data enables the method to overcome the limitations of self-reporting alone, such as recall bias or underreporting, and enhances a reliability of the stress assessment. Furthermore, by deriving the stress reaction, the method achieves a dynamic and context-aware understanding of the stress, rather than a static or isolated measurement. The validation of the stress reaction using longitudinal stress reaction data further strengthens the method's accuracy, as it accounts for individual variability and temporal trends, distinguishing between acute and chronic stress patterns. The modelling of the stress level from the stress reaction derived enables flexible and robust stress quantification, adaptable to different user scenarios and data availability. This approach supports both immediate feedback and long-term monitoring, empowering users and healthcare providers to make informed decisions regarding stress management, intervention, and recovery strategies.

In a second aspect, the present disclosure provides a system for defining a stress level, the system comprising a processor configured to:
obtain, via a user interface communicably coupled to the processor, a first set of information related to mental stress from a user via a first questionary;
obtain, via a user interface, a second set of information related to physical stress from the user via a second questionary;
receive a hormonal level, measured using a sensor arrangement, at a first moment of time;
use the first and second sets of information and the measured hormonal level to derive a stress reaction over a pre-defined period of time;
validate the stress reaction using longitudinal stress reaction; and
define the stress level by modelling stress level from at least one of:
   the stress reaction from at least one of: the first and second questionaries;
   the validated stress reaction; and
display, on the user interface, the stress level as a visual representation.

The present disclosure provides the aforementioned system to facilitate an accurate, efficient, and real-time detection and monitoring of an individual's stress level by integrating subjective and objective data sources. The system provides actionable insights that support personal health management, stress reduction interventions, and long-term well-being. The acquisition of mental and physical stress information via user-friendly questionnaires ensures that the system captures the user's subjective experience, which is essential for personalized stress assessment. The integration of hormonal measurements, obtained through the sensor arrangement, adds an objective, physiological dimension to the analysis, reducing bias and enhancing the reliability of the stress evaluation. The processor's capability to derive the stress reaction enables dynamic and context-aware stress modelling. The validation of the stress reaction using longitudinal data further improves accuracy by accounting for individual variability and temporal trends, distinguishing between acute and chronic stress responses.

By modelling the stress level from either the immediate stress reaction, the validated longitudinal reaction, or both, the system provides flexible and robust stress quantification tailored to different use cases and user needs. The visual representation of the stress level on the user interface transforms complex data into intuitive feedback, empowering users to understand and manage their stress in real time. This enables early intervention, supports mental and physical health optimization, and facilitates ongoing self-care and professional guidance. The system is suitable for integration into wellness platforms, occupational health programs, and clinical environments, providing scalable and individualized stress management solutions.

Throughout the present disclosure, the term “stress level” refers to a quantified or modelled representation of an individual's overall stress state derived from multiple dimensions of stress. Notably, the stress level is a measurable parameter that reflects intensity, quantity and impact of the stress on the individual over a specific period of time. It will be appreciated that the method addresses the limitations of existing stress management techniques which often focus on isolated aspects of the stress (e.g., mental stress, physical exertion, hormonal levels and the like) by integrating mental, physical, and hormonal data to provides a complete picture of the stress. A technical effect of the method is a significant improvement in stress management, enabling better stress management and health outcomes.

Throughout the present disclosure, the term “mental stress” refers to a psychological strain or a pressure experienced by the user due to cognitive or emotional demands. Notably, the mental stress is a subjective state influenced by factors such as workload, cognitive emotional challenges, and the user's ability to cope with stressors.

Optionally, the mental stress comprises:
a set of mental load to be selected by the user on a scale of 0 to 10; and
a plurality of tags indicative of a user's perception of the mental load, wherein each tag has a score on a scale of 0 to 5, to be selected by the user.

In this regard, the term “mental load” refers to the amount of mental workload that a user is currently experiencing or has endured. The mental load is quantified by asking the user to rate their perceived mental workload on a scale from 0 to 10. The set of mental load is the collection of mental load values provided by the user over time or during a specific session. The set of mental load is used to analyze trends and patterns in the user's mental workload. The set of mental load may, for example, be selected by the user on the scale of 0, 1, 2, 3, 4, 5, 6, 7, 8 or 9 up to 1, 2, 3, 4, 5, 6, 7, 8, 9 or 10. For example, the user rates the mental load at the end of each day for a week, such as DAY 1: 6 (moderate workload, so some stress), DAY 2: 8 (high workload, significant stress), DAY 5: 4 (low workload, minimal stress). The set of mental load may include 6, 8 4. In another example, the user rates their mental load at different times during a single workday, such as morning (9:00 AM): 7 (High workload, preparing for meetings), midday (12:00 PM): 6 (Moderate workload, handling tasks), afternoon (3:00 PM): 8 (High workload, meeting deadlines), evening (6:00 PM): 5 (Moderate workload, winding down) and the set of mental load 7, 6, 8, 5 is selected. The aforementioned examples illustrate how a set of mental load can be collected over different timeframes or scenarios, providing valuable data for analyzing trends, patterns, and variations in the user's mental workload. The term “tag” refers to predetermined descriptors selected by the user from predefined baskets of stress-related and recovery-related options. The plurality of tags are selected by the user from a basket of stress-related and recovery-related tags that are used to gauge the user's perception of their mental state and how well the user is coping with the mental load. The term “user's perception” refers to a subjective assessment of the user's mental state, as reflected in the mental load and the tags selected by the user. The term “score” refers to a numerical value assigned to each tag, which can range from 0 to 5. The tag that has a score on the scale 0, 1, 2, 3 or 4 up to 1, 2, 3, 4 or 5 may be selected by the user. The score represents the impact of the plurality of tags on the user's mental state, with higher scores indicating a greater influence. It will be appreciated that the set of mental load provides a numerical representation of the stress, while the plurality of tags add context and depth by capturing the user's perception of their mental state. By using the plurality of tags with associated scores, the method minimizes bias and variability in user responses. A technical effect of the aforementioned clause is to ensure that the combination of mental load and the tags allows for a more nuanced understanding of mental stress, capturing both the intensity and the user's emotional or cognitive response to stressors.

Optionally, the plurality of tags comprises:
negative tags, associated with stress, selected from at least one of: good resources, relaxation, comfortable, fulfilment, flow state; and
positive tags, associated with recovery, selected from at least one of: pressure to perform, nervousness, irritation, task load, aggravated.

In this regard, the term “negative tags” refers to descriptors that are associated with stress and represent factors contributing to the user's mental workload or emotional strain. The negative tags may include good resources, relaxation, comfortable, fulfillment, flow state and the like. The term “relaxation” refers to a negative tag that represents the user's perception of being in a calm and stress-free state. The term “flow state” refers to a negative tag that represents the user's perception of being fully immersed and engaged in an activity, often leading to a sense of enjoyment and reduced stress. The negative tags are indicative of recovery-related states that help mitigate or counteract the effects of stress. For example, the negative tags such as “relaxation” and “flow state” represent conditions that reduce the user's mental workload and promote recovery. It will be appreciated that the user selects one or more negative tags from a predefined basket of options. Each negative tag is associated with a positive score, as the negative tags represent recovery-related states that reduce the user's overall stress level. The term “positive tags” refers to descriptors that are associated with the recovery and represent factors contributing to the user's mental workload or the emotional strain. For example, the positive tags such as “pressure to perform” and “nervousness” represent conditions that elevate the user's stress level. The term “pressure to perform” refers to a positive tag that represents the user's perception of being under stress to meet expectations or achieve goals. The pressure to perform tag is associated with a negative score, as it indicates a stress-related state that increases the user's overall stress level. The nervousness represents the user's perception of feeling anxious or uneasy. The irritation represents the user's perception of being annoyed or frustrated. The term “task load” refers to a positive tag that represents the user's perception of having a high volume of tasks or responsibilities. The term “aggravated” refers to a positive tag that represents the user's perception of being increasingly stressed or frustrated. The user selects one or more positive tags from a predefined basket of options. Each positive tag is associated with a negative score, as these tags represent stress-related states that increase the user's mental workload. It will be appreciated that by capturing recovery-related and stress-related states, the plurality of tags provides a complete picture of the user's mental state. A technical effect of incorporating the negative tags and the positive tags ensures that method captures the user's perception of both recovery-related and stress-related states, providing a complete picture of mental stress and reduces subjectivity in the assessment process. Additionally, an improved ability to measure and interpret the mental stress, enabling better stress management and personalized interventions for the user.

The term “first set of information” refers to data collected from the user that is specifically related to the mental stress of the user. The first set of information may include numerical ratings (e.g., stress level on a scale of 0 to 10), qualitative inputs (e.g., tags or descriptors), contextual data (e.g., nature of the stressor) and the like that reflect the user's mental workload, emotional state, and perceived stress levels. The first set of information may also include the user's self-reported input and serve as a foundation for assessing the user's mental stress. The term “user” refers to an individual that provides the data related to their mental stress. For example, the user may be a person seeking to monitor and manage their stress levels, an individual participating in a wellness or health program, a subject in research or a clinical study related to the stress, and the like. The term “first questionary” refers to a structured set of questions that is designed to collect the first set of information related to the mental stress. The user is a subject of the stress analysis and provides necessary data for the method to define a stress level. The first questionary serves as a tool or an interface through which the first set of information is obtained. The first questionary may include quantitative questions (e.g., asking the user to rate their stress level on a numerical scale), qualitative questions (e.g., asking the user to select the tags or the descriptors that best represent their emotional state or the stressors), contextual questions (e.g., source of stress or the user's coping mechanism) and the like. It will be appreciated that by using the first questionary to obtain the first set of information, the method ensures that the mental stress is quantified in a structured and reliable manner.

Throughout the present disclosure, the term “second set of information” refers to data collected from the user that is specifically related to their physical stress. It will be appreciated that the second set of information is distinct from the first set of information, which is related to the mental stress of the user. For example, the second set of information may include information about the parameters (such as duration of physical tasks), duration of tasks performed under various conditions and the like. The second set of information is used to quantify and analyze the user's physical stress levels, providing an objective basis for assessing physical exertion. The term “physical stress” refers to a physiological strain experienced by the user due to the physical exertion. For example, the physical stress may include fatigue and exhaustion. The physical stress is characterized by measurable parameters such as heart rate, hormonal levels, duration of physical tasks, and the like. The term “second questionary” refers to a structured set of questions that is designed to collect the second set of information related to the physical stress of the user. The second questionary serve as a tool or an interface to obtain the second set of information related to user's physical exertion. For example, the second questionary may include questions related to the duration and intensity of physical activities, contextual questions about the user's physical workload and capacity, specific metrics (such as the duration of tasks performed under high physical strain), and the like. The second questionary complements the first questionary to provide a holistic view of the user's stress levels. The physical stress is assessed using the second set of information and the second questionary. It will be appreciated that the process of obtaining the second set of information related to the physical stress of the user enables quantification of the physical stress which is often subjective and difficult to measure accurately without structured data collection. The second set of information collected through the second questionary provides objective insights into the user's physical workload and capacity, which are essential for stress management and recovery planning.

Optionally, the second questionary is associated with a set of physical stress parameters comprising at least one of:
a duration of performing physical task;
a duration of performing physical task when the user is not able to speak more than one word; and
a duration of performing physical task until exhaustion.

In this regard, the term “set of physical stress parameters” refers to a collection of measurable attributes or metrics that are used to quantify the physical stress experienced by the user. The set of physical stress parameters serves as a framework for assessing the user's physical workload and exertion levels. Each parameter within the set of physical stress parameters provides a unique perspective on the user's physical stress. The set of physical stress parameters is directly associated with the second questionary, which is designed to collect data related to these parameters. The phrase “duration of performing physical task” refers to a total time spent by the user in completing a specific physical activity or task (such as how long the user exercises or did a physically demanding activity). The duration of performing the physical task provides a baseline measure of the user's physical workload. The phrase “duration of so performing physical task when the user is not able to speak more than one word” refers to a time period during which the user is performing a physical task at an intensity level that limits the user's ability to speak more than one word at a time. The parameter provides an intermediate measure of physical stress that may precede the exhaustion. The parameter is an indicator of high physical exertion and is used to assess the intensity of the user's physical stress. The term “exhaustion” refers to a state in which the user is no longer able to continue performing a physical task such as not able to finish a sentence due to physical fatigue or depletion of energy reserve. The exhaustion represents an upper limit of the user's physical capacity. The duration of performing physical task until exhaustion is a parameter within the set of physical stress parameters, as it quantifies the user's endurance and physical capacity. It will be appreciated that the reason for including the set of physical stress parameters is to provide a simple, user-friendly and effective way to quantify the physical stress without a need for complex equipment (such as sports computer or continuous heart rate monitors). The set of physical stress parameters allows for differentiation between moderate and high-intensity efforts, which have different physiological and hormonal impacts. A technical effect of associating the second questionary with the set of physical stress parameters is to ensure a detailed and accurate evaluation of the physical stress. Additionally, the second questionary allows for making tailored stress management strategies based on the user's unique physical stress profile.

Throughout the present disclosure, the term “hormonal level” refers to a quantitative measurement of specific hormones in the user's body that are indicative of stress and recovery states. It will be appreciated that primary hormones that are being assessed are cortisol and testosterone. The hormonal level is measured using a biological sample (such as saliva, blood, or another suitable medium). The term “first moment of time” refers to a point in time at which the hormonal level is measured. The first moment of time is an initial time reference for capturing the user's physiological state, typically, before or at the start of a period of interest (for example, before a stress event, after waking, or at a schedule assessment time). The first point of time serves as a baseline or starting reference for subsequent analysis, such as tracking changes in hormonal levels over time. Moreover, the purpose of measuring the hormonal level at the first moment of time is to obtain an objective biomarker of stress and recovery, which complements subjective data collected via the first questionary and the second questionary. At the first moment of time, a biological sample is collected from the user. The biological sample is analyzed to determine the concentration of relevant hormones. The measured hormonal level is recorded and used as a data point in the overall stress assessment model.

Optionally, the step of measuring the hormonal level at the first moment of time uses dual markers for stress and anabolism, and wherein the dual markers are selected from:
a stress marker comprising cortisol (C); and
an anabolic marker comprising testosterone (T).

In this regard, the term “stress marker” refers to a hormone whose concentration in the body of the user increases in response to the physical or the mental stress. It will be appreciated that the stress marker is cortisol (C). The cortisol level indicates the presence and magnitude of the stress in the user body. The term “anabolic marker” refers to a hormone that reflects the body's anabolic (such as building and recovery) state. In this context, the anabolic marker is testosterone (T). The testosterone indicates the body's increased capacity for tissue repair, muscle growth, and overall recovery from the stress. The term “anabolism” refers to a recovery and restorative process of the user's body that is promoted by anabolic hormones such as testosterone. Herein, the dual markers refers to two different types of hormones (such as cortisol and testosterone) in the user's body. The dual markers are measured together to provide a comprehensive picture of the user's physiological state. The cortisol serve as a marker for the stress, and the testosterone serve as a marker for the anabolism. High cortisol with low testosterone may indicate harmful, chronic stress, while high testosterone (even with elevated cortisol) may indicate good adaptation or recovery. Moreover, by measuring the dual markers at the same time, the method is able to assess not only the presence of stress but also the body's capacity for recovery and adaptation. It will be appreciated that the markers allows the method to distinguish between harmful (such as catabolic) stress and beneficial (anabolic) recovery, enabling more precise and actionable stress management. A technical effect of using the dual markers for stress and anabolism is to ensure that the method is able to distinguish between “bad” stress (high cortisol, low testosterone) and “good” stress or recovery (high testosterone, balanced or even elevated cortisol). Additionally, the dual markers accurately reflect the user's physiological state, reducing the risk of false positives or negatives in stress assessment.

Throughout the present disclosure, the term “stress reaction” refers to a physiological and a psychological response of the user to stressors. Typically, the stress reaction reflects how the user's body and mind are responding to the stress at a given time and how this response evolves over the time. The stress reaction is quantified using various metrics, such as the magnitude of change in the stress and the anabolic hormone levels over the pre-defined period of time, balance or ratio between the cortisol and the testosterone, and the like. The stress reaction of the user is derived by integrating the first and the second set of information and measured hormonal level. The term “pre-defined period of time” refers to a predetermined time interval over which the stress reaction of the user is assessed and analyzed. The pre-define period of time is set in advance and it can vary depending on the application or user needs. For example, the pre-defined period of time may include a single day, several consecutive days, duration of a specific event or an activity, and the like. The derived stress reaction is represented numerically, graphically, or as a classification (for example, high/low stress). The purpose of defining said period is to enable consistent tracking of how the stress reaction of the user develops, persists or resolves over time. It will be appreciated that the integration of the first and the second set of information, and the measured hormonal level reduces the risk of bias or error that may occur when relying on a single type of measurement. By tracking the stress reaction over the predefined period of time, the method facilitates the user and healthcare providers to identify harmful stress patterns early and intervene more effectively.

Optionally, the step of deriving the stress reaction further comprises:
scaling the mental load to produce an adjusted mental load, using a relation

$${Mental}_{adj} = ( \frac{Mental load - 1}{9} ) * 10 ;$$

computing a flow score from the selected tags, wherein the flow score is calculated by subtracting the sum of the tag scores from a predetermined baseline value using a relation

$$Flow score = 5 - ( sum of tag scores ) ;$$

weighting the adjusted mental load with the flow score to determine an initial stress level using a relation:

$$Initial Stress Value = {Mental}_{adj} * ( \frac{10 - Flow score}{5} ) ;$$

and
applying a transformation function to the initial stress level such that the transformed stress level lies within a normalized range using a relation

$$T ( X ) = ( - 1 + \frac{1 + 1}{1 + {e}^{- 0.22 * 10 * ( \frac{X}{10} )}} ) * 10 .$$

In this regard, the term “scaling” refers to a mathematical process of converting the mental load, as originally reported by the user on a scale from 0 to 10, into a new value that is distributed over a different range. Scaling is performed using the following relation

$${Mental}_{adj} = ( \frac{Mental load - 1}{9} ) * 10$$

Where the mental load is a user input. The scaling ensures that a minimum mental load value maps to 0 and the maximum value maps to 10, thus producing the adjusted mental load. The term “adjusted mental load” refers to an adjusted value of the user's perceived mental load, ensuring comparability across different users and sessions. Notably, the adjusted mental load is a result of the scaling operation applied to the mental load. The term “flow score” refers to a cumulative score computed from the tags selected by the user. The term “selected tags” refers to specific descriptors selected by the user from the set of positive and negative tags. Each selected tag amongst the plurality of tags has an associated score. The flow score is calculated by subtracting the sum of tag scores from the predetermined baseline value. The term “predetermined baseline value” refers to a constant value from which the sum of the tag scores is subtracted to compute the flow score. For example, the predetermined baseline value is 5. The predetermined baseline value serves as a reference point, ensuring that the flow score reflects deviations from a standard or expected level of coping ability. The flow score is calculated using the following relation

$$Flow score = 5 - ( sum of tag scores )$$

The flow score quantifies the user's subjective perception of their ability to cope with the mental load. The term “initial stress level” refers to a preliminary quantification of the user's stress, calculated by weighting the adjusted mental load with a factor derived from the flow score. The initial stress value is determined using the following relation

$$Initial Stress Value = {Mental}_{adj} * ( \frac{10 - Flow score}{5} )$$

The initial stress value integrates both the adjusted mental load and the user's perceived coping ability, as reflected by the flow score. The term “transformation function” refers to a mathematical function applied to the initial stress value to map the initial stress value onto a normalized range, typically from 0 to 10. The transformation function is defined by the following relation

$$T ( X ) = ( - 1 + \frac{1 + 1}{1 + {e}^{- 0.22 * 10 * ( \frac{X}{10} )}} ) * 10$$

Where X is the initial stress value. The transformation function T ensures that the final stress value is both intuitively understandable and bounded within the desired range to facilitate interpretation. A technical effect of the aforementioned feature is to ensure that the final stress value is within a normalized range. Additionally, it ensures enhanced accuracy and reliability in quantifying the mental stress by mathematically integrating both the intensity of the mental load and the user's subjective coping. Normalization and comparability of stress values across different users and time periods.

Optionally, the transformation function enables transforming the stress level to be measured on a scale of 1 to 10. In this regard, the transformation function is designed to convert the stress level (which may initially be on an arbitrary scale) into a standardized and normalized scale ranging from 1 to 10. The stress level may be transformed on the scale from 1, 2, 3, 4, 5, 6, 7, 8 or 9 up to 2, 3, 4, 5, 6, 7, 8, 9 or 10. It will be appreciated that the transformation function mathematically maps the input stress value to said fixed interval, ensuring that all final stress scores are expressed within said range. The transformation of the stress level makes it easier for the users to assess their own stress and track changes over time. The transformation function maps the lower input values linearly or non-linearly in the range of [0,5]. Higher input values are compressed or tapered off in the range of [5,10] so that the output never exceeds 10, regardless of how high the input is. A technical effect of the aforementioned feature is to ensure consistency and interpretability of the stress measurement system, as the users and the practitioners are able to quickly understand and act on results presented on the 1-10 scale.

Optionally, the transformation function is a generalized logistic function. In this regard, the term “generalized logistic function” refers to a mathematical tool that ensures all calculated stress levels are normalized to a standard, bounded, and interpretable scale. The generalized logistic function is represented as the following equation.

$$T ( X ) = ( - 1 + \frac{1 + 1}{1 + {e}^{- 0.22 * 10 * ( \frac{X}{10} )}} ) * 10$$

The generalized logistic function is chosen as the transformation function to ensure that, regardless of the initial stress level's magnitude, the final stress level is always mapped to a value between 1 and 10. A technical effect of the aforementioned feature is to improve usability for end-users and health professionals by providing a familiar and actionable range.

Throughout the present disclosure, the term “longitudinal stress reaction” refers to an observation and assessment of the stress responses over an extended period to observe trends, patterns, and recovery processes. Notably, the longitudinal stress reaction serve as a reference for validating the accuracy and reliability of the stress reaction derived over the pre-defined period of time. The validation is performed by comparing the derived stress reaction with the longitudinal stress reaction. Moreover, the longitudinal stress reaction tracks the evolution of the stress marker and the anabolic marker across multiple time points (e.g., daily, weekly and the like). It will be appreciated that the validating the stress reaction using the longitudinal stress reaction ensures that the derived stress reaction is consistent with the user's actual physiological trends, as observed in the longitudinal stress reaction.

Optionally, the step of longitudinal stress reaction is based on the hormonal level. In this regard, the process of assessing the longitudinal stress reaction is performed using the hormonal level. The step of longitudinal stress reaction relies on measuring the concentrations and ratios (T/C or C/T) of the hormones such as cortisol (the stress marker) and testosterone (the anabolic or recovery marker), which act in opposite directions in the body's stress response, at multiple time instants to observe how the stress and the recovery evolve longitudinally over the time. It will be appreciated that the hormonal level provide direct, quantifiable evidence of the body's stress and recovery states, which are less susceptible to subjective bias than self-reported measures. This approach enables analysis of both the initiation and magnitude of a stress event. A technical effect of the aforementioned feature is to improve accuracy by analyzing the stress assessment with physiological data rather than subjective reports alone. Additionally, it supports personalized interventions by revealing individual patterns of stress and recovery, allowing for tailored recommendations.

Throughout the present disclosure, the stress level of the user is defined by constructing a model for the stress level that draws on the stress reaction calculated from the mental and physical stress questionnaires, and from the validated stress reaction, through longitudinal hormonal data. It will be appreciated that by utilizing the first and the second questionaries, and the validated stress reaction, the method enhances the reliability and personalization of stress monitoring. For example, by combining the levels of mental stress, physical stress and hormonal responses and how they change day-to-day, the method able to analyze and give interpretation to the different combinations of parameters, their levels and days. The modelling of the stress level uses the direct output from the first and the questionaries, and the validated stress reaction to define the user's current stress level, ensuring the most accurate and appropriate assessment is used. This modeling of the stress level supports continuous, real-time feedback when only questionnaire data is available, while also enabling higher-fidelity validation when physiological data is present.

Optionally, the method further comprises hormone-based stress ignition-consequence tracking based on the hormonal level over the predefined period of time, wherein the hormonal levels comprise: a stress marker, and an anabolic marker. In this regard, the term “hormone-based stress ignition-consequence tracking” refers to a dynamic, hormone driven monitoring process that captures both an onset and an aftermath of stress events by analyzing changes in the stress and the anabolic hormone levels over the pre-defined period of time. Herein, the process involves two phases, one is hormone-based stress ignition, and the other is consequence tracking. The hormone-based stress ignition is an initial response of the user's body to the stressor. The initial response is identified by a measurable change in the stress marker (e.g., a spike in cortisol concentration) and/or shift in the balance between the stress marker and the anabolic marker (e.g., a decrease in the testosterone/cortisol (T/C) ratio). This phase captures not only the immediate reaction to the stress, but also the magnitude and quality of the recovery phase. Following the hormone-based stress ignition, the method tracks the evolution of both the marker (i.e. the stress marker and the anabolic marker) over the pre-defined period of time. The consequence tracking includes monitoring the duration and area under the curve of elevated or suppressed hormone levels, as well as the recovery trajectory indicated by the anabolic marker. The process reveals how quickly and effectively the body returns to baseline or compensates after the stress event. It will be appreciated that the hormone-based stress ignition-consequence tracking allows to pinpoint an exact moment and intensity of stress initiation using absolute hormone concentrations and their ratios. Additionally, it allows for evaluating quality of recovery, distinguishing between beneficial (adaptive) and detrimental stress based on the compensatory response of the anabolic marker. A technical effect of the aforementioned feature is to enable a comprehensive understanding of not just when and how strongly the stress occurs, but also how well the body recovers.

Optionally, a hormone-based stress ignition is indicated by an absolute concentration of the stress marker, wherein the stress marker comprises cortisol; and
a magnitude of the hormone-based stress ignition is indicated by a change in equilibrium of concentrations of the stress marker and the anabolic marker, wherein the anabolic marker comprises testosterone (T).

in this regard, the term “hormone-based stress ignition” refers to a physiological event that marks the onset of the stress response, as detected through hormonal analysis. The detection of hormone-based stress ignition is not based on subjective symptoms or behavioral cues, but rather on the direct measurement of the hormone levels. The term “absolute concentration” refers to an actual, quantitative amount of the stress marker (cortisol) present in the biological sample at a specific point in time that is used to pinpoint the ignition event. Typically, the absolute concentration is expressed in standard units (e.g., nanomoles per liter, nmol/L). The change in equilibrium of concentrations of the stress marker and the anabolic marker is characterized by observing how the balance between cortisol (stress marker) and testosterone (anabolic marker) shifted the body's hormonal balance, typically using ratios such as T/C or C/T, provides the measure of the magnitude of the stress event. The method measures the absolute concentration of the cortisol. If the concentration of the cortisol crosses a certain threshold, it indicates that a stress response is triggered (namely, ignition). A technical effect of the aforementioned feature is to ensure dynamic tracking of the stress responses over time, rather than a static, one-time measurement, which is valuable for applications in health monitoring, sports science, and personalized medicine. Additionally, it is able to more accurately distinguish between normal and stress states.

Optionally, a hormone-based stress ignition-consequence is indicated by a area under curve of the change in equilibrium of the concentrations of the stress marker and the anabolic marker; and
a recovery of resources and healing processes is indicated by a concentration of the anabolic marker.

In this regard, the term “area under curve” refers to an integral or total value of consequence of the hormone-based stress ignition accumulated over time of the change in equilibrium between the concentrations of the stress marker (cortisol) and the anabolic marker (testosterone). Typically, the area under curve quantifies the overall impact of the stress event over a period, not just at a single time instant because stress events are not just momentary. The area under curve captures both the magnitude and duration of a deviation in the hormonal balance caused by the stress. A larger area under curve indicates a more significant or prolonged stress consequence. The phrase “recovery of resources” refers to a physiological process by which the body restores its balance and returns to a non-stressed, anabolic state after the stress event. The rediscovery is indicated by the concentration of the anabolic marker (testosterone). When the concentration of testosterone returns to or exceeds a certain threshold, it signals that the body is recovering, rebuilding, and engaging in healing processes after the stress response. The concentration refers to a recovered or an overcompensating anabolic marker. A technical effect of the aforementioned feature is to enable personalized health management, as it can adapt to individual baseline hormone levels and provide actionable insights for interventions or lifestyle adjustments.

Optionally, the concentration of the anabolic marker is indicative of a good stress or a bad stress, and wherein the bad stress is indicated by higher stress marker and good stress is indicated by a higher anabolic marker. In this regard, the term “good stress” refers to a stress event that, after the initial stress response, is followed by a higher concentration of the anabolic marker (such as testosterone). The good stress indicates that the body has not only recovered from the stressor but has also entered a state of anabolic (building, healing, or strengthening) activity. For example, the user experiences chronic work stress (high cortisol). When the user engages in a positive stressor, such as exercise or an ice bath, there is an initial spike in the stress marker (cortisol), but this is followed by a strong increase in the anabolic marker (testosterone). This rebound or overcompensation in the anabolic marker indicates effective recovery and adaptation. The term “bad stress” refers to a stress event or condition where the concentration of the stress marker (such as cortisol) remains high and/or the anabolic marker (such as testosterone) does not sufficiently recover or remains low. The bad stress indicates that the body is not effectively recovering, leading to prolonged breakdown states, resource depletion, or impaired healing. The bad stress is associated with negative health consequences, such as chronic fatigue, poor adaptation, or increased risk of illness. For example, during illness (flu), the user shows a prolonged and high stress marker (cortisol) and a decreased anabolic marker (testosterone/cortisol ratio, T/C). A technical effect of the aforementioned feature is to ensure that by distinguishing the good stress from the bad stress, interventions (like rest, training adjustments, or medical attention) is better targeted as per the user's requirement.

Optionally, the method further comprises storing, in a database, responses to the obtained first and second questionaries and hormonal levels. In this regard, the term “database” refers to a structured storage system or repository that is configured to store the responses to the obtained first and second questionaries and hormonal levels in the structured or the unstructured format. The database me be local or cloud-based database. The purpose of storing the responses to the obtained first and second questionaries and hormonal levels in the database is to enable long-term tracking, analysis, and retrieval of user data by an authorized user. By keeping the said information in the database, the method is able to monitor changes and trends in the stress and the recovery over time, ensure data is available for future reference, audits, or research, and the like. A technical effect of the aforementioned feature is to ensure that the said information is organized, secured, and readily available for authorized use, supporting compliance and user trust. Additionally, the database can be used to train algorithms for more sophisticated modeling and prediction of the stress responses.

Optionally, the method further comprises employing a deep neural network (DNN) algorithm trained on a population data set to model user-specific stress patterns and to refine the derivation of the stress level. In this regard, the term “deep neural network (DNN) algorithm” refers to a type of artificial intelligence (AI) model inspired by the structure and function of the human brain that is used to analyze and learn from questionnaire responses and hormonal measurements to identify stress patterns and relationships that can predict or model user-specific stress responses. The term “population data set” refers to a large collection of data gathered from many individuals representing a diverse group of users. The population data set includes responses to the first and the second questionaries, hormonal measurements, and the like. The population data set is used to train the DNN algorithm. By learning from a wide range of real-world examples, the DNN algorithm is able generalize and accurately model stress patterns, not just for the general population but also for individual users by comparing their data to the broader trends. It will be appreciated that by leveraging the DNN algorithm and learning from a large population data set, the method increases accuracy, personalization and predictive power of the stress assessment. The method is able to recognize subtle and complex patterns in the stress data and adapt to individual differences. The trained DNN algorithm is applied to the user data, allowing the method to model that user's unique stress patterns and refine the calculation or prediction of their stress level. A technical effect of the aforementioned feature allows for automated, large-scale deployment without manual tuning for each user that increases efficiency. Additionally, The DNN can be retrained to reflect new trends, health insights, or population changes, keeping the system up to date.

Optionally, the stress level is visually represented in any of: a tabular form, a graphical form. In this regard, the term “tabular form” refers to a form of visually representing the stress level data in a structured table format. In the tabular format, information is organized into rows and columns, where each row represents a specific data entry (such as a day, event, or measurement), and each column represents a particular parameter (such as date, stress level, cortisol value, testosterone value, etc.). The term “graphical form” refers to a way of visually representing the stress level data using charts, graphs, or other visual elements. The graphical form may include line graphs, bar charts, scatter plots, or other types of visualizations that display trends, patterns, or relationships in the data over time or across different conditions. It will be appreciated that visual representation of the stress level in said forms makes the stress data easily understandable. The stress level is displayed, in the said forms, on a user interface, so that the user is able to easily review and interpret the stress information. A technical effect of the aforementioned feature is to ensure that the visual formats facilitate the user and clinician spot important changes or issues at a glance, supporting timely interventions. Additionally, trends and patterns over time are easier to see in the visual formats.

The present disclosure also relates to the system as described above. Various embodiments and variants disclosed above, with respect to the aforementioned method, apply mutatis mutandis to the system.

Throughout the present disclosure, the term “processor” refers to a computational element that is operable to execute the software framework. Examples of the processor may include, but are not limited to, a microprocessor, a microcontroller, a complex instruction set computing (CISC) microprocessor, a reduced instruction set computing (RISC) microprocessor, a very long instruction word (VLIW) microprocessor, or any other type of processing circuit. The processor is configured to collect user inputs, receive sensor data from the sensor arrangement, perform stress analysis, and manage data flow between system components. The processor is essential for coordinating the flow of data and ensuring that each component operates in sequence to define the stress level.

Throughout the present disclosure, the term “user interface” refers to an interface that encompasses elements and components through which a user interacts with a computer-related application within the computing device. The user interface may include graphical user interfaces (GUIs) on devices like smartphones, tablets, or computers, as well as physical interfaces such as touchscreens, buttons, or voice input. The user interface is used to present the first and the second questionaries to the user, collect their responses, and display the calculated stress level and related information. The term “sensor arrangement” refers to an arrangement of one or more sensors that measures physiological parameters, for example, hormonal levels (such as cortisol or testosterone) from biological samples (e.g., saliva, blood, or other fluids). The sensor arrangement may include biosensors, lab-on-chip device and the like biochemical sensors, and any associated electronics required to capture and transmit the measurement data to the processor.

Optionally, the system further comprises a database configured to store responses to the obtained first and second questionaries and hormonal levels.

Optionally, the system further comprises a deep neural network (DNN) algorithm trained on a population data set to model user-specific stress patterns and to refine the derivation of the stress level.

The present disclosure also relates to the computer program product as described above. Various embodiments and variants disclosed above, with respect to the aforementioned method and the aforementioned system, apply mutatis mutandis to the computer program product.

### DETAILED DESCRIPTION OF THE DRAWINGS

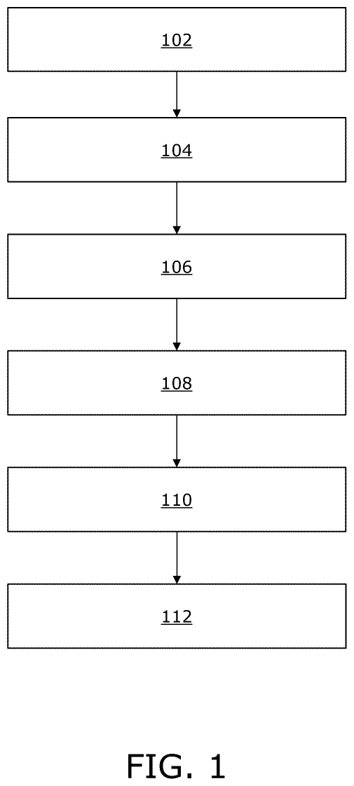

Referring to FIG. 1, illustrated is a flow chart depicting steps of a method for defining a stress level, in accordance with an embodiment of the present disclosure. At step 102, a first set of information related to mental stress is obtained from a user via a first questionary. At step 104, a second set of information related to physical stress is obtained from the user via a second questionary. At step 106, a hormonal level is measured at a first moment of time. At step 108, a stress reaction is derived using the first and second sets of information and the measured hormonal level over a pre-defined period of time. At step 110, the stress reaction is validated using longitudinal stress reaction. At step 112, the stress level is defined by modelling stress level from at least one of: the stress reaction from at least one of: the first and second questionaries; the validated stress reaction.

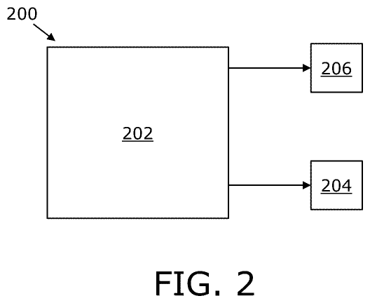

Referring to FIG. 2, illustrated is a block diagram of a system 200 for defining a stress level, in accordance with an embodiment of the present disclosure. As shown, the system 200 comprises a processor 202. The processor 202 is configured to obtain, via a user interface 204 communicably coupled to the processor, a first set of information related to mental stress from a user via a first questionary. Moreover, the processor 202 is configured to obtain, via a user interface, a second set of information related to physical stress from the user via a second questionary. Furthermore, the processor 202 is configured to receive a hormonal level, measured using a sensor arrangement 206, at a first moment of time. Furthermore, the processor 202 is configured to use the first and second sets of information and the measured hormonal level to derive a stress reaction over a pre-defined period of time. Furthermore, the processor 202 validate the stress reaction using longitudinal stress reaction. Furthermore, the processor 202 is configured to define the stress level by modelling stress level from at least one of: the stress reaction from at least one of: the first and second questionaries; the validated stress reaction. Furthermore, the processor 202 is configured to display on the user interface 204, the stress level as a visual representation.

Referring to FIG. 3, illustrated is an exemplary implementation 300 of a stress level analysis, in accordance with an embodiment of the present disclosure. As shown, FIG. 3 presents real-life data of day-to-day evening salivary samples, showing the acute accumulation of day-stress. The patterned bars represent the normalized cortisol value (as a percentage of the user's mean), while the plain bars represent the normalized T/C (testosterone/cortisol) value. The figure illustrates how acute stress and recovery are reflected in hormonal markers, with notable events such as illness (flu) and changes in exercise modality (from running to biking) visible in the hormonal response patterns.

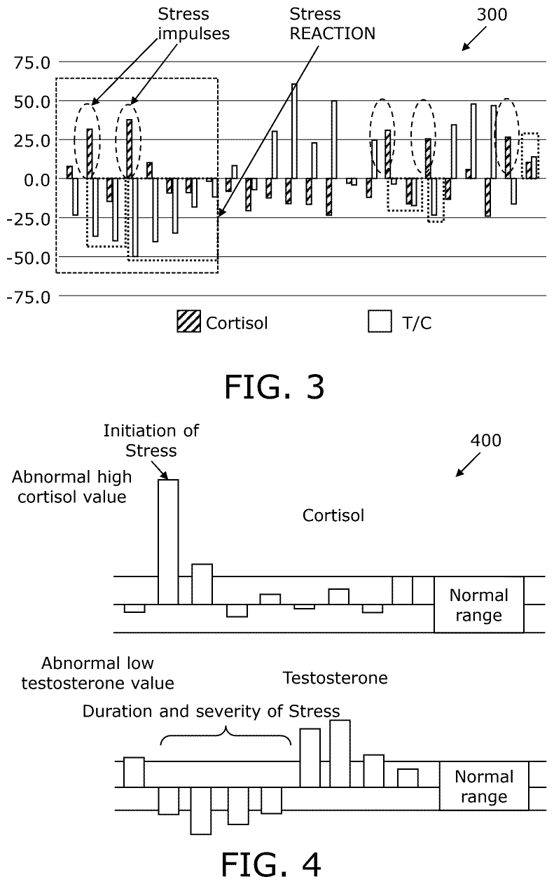

Referring to FIG. 4, illustrated is a graphical representation 400 of stress level analysis, in accordance with an embodiment of the present disclosure. FIG. 4 provides a breakdown of the roles of the stress hormone (cortisol (C)) and the anabolic hormone (testosterone (T)), as a series of daily evening samples. The figure highlights that simply observing the withdrawal of stress-Initiated cortisol is insufficient. The implementation 400 emphasizes the need to also monitor recovery processes, as indicated by anabolic markers, to fully understand the duration and magnitude of stress consequences.

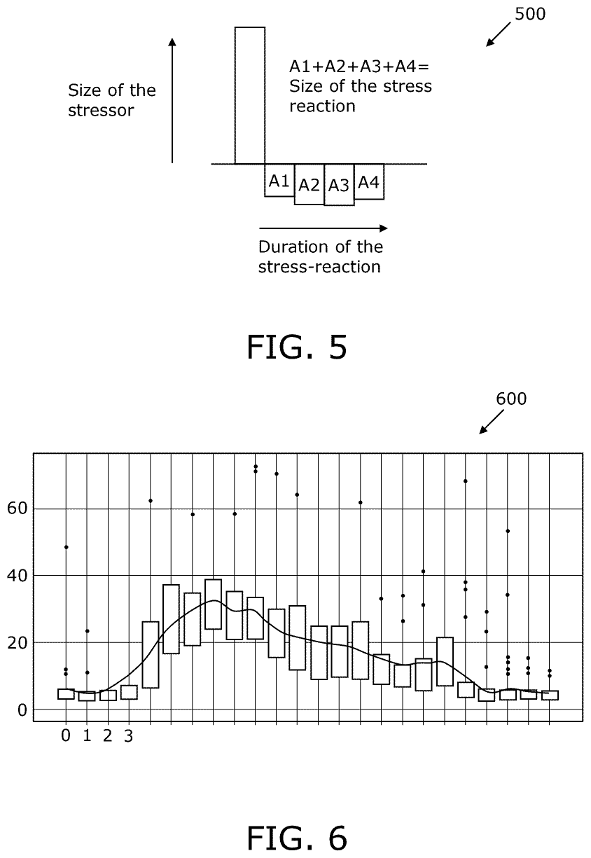

Referring to FIG. 5, illustrated is a schematic illustration of a graph 500 for assessing the duration and magnitude of a stress reaction, in accordance with an embodiment of the present disclosure. As shown in the figure, the X-axis denotes the duration of the stress reaction, and the Y-axis denotes the size of the stressor. The first bar represents the stress-initiation, depicted as (C) in the figure, and the subsequent bars show the stress reaction as measured by the T/C ratio. A more negative value in a subject-normalized graph indicates a higher stress reaction. The figure also suggests that the stress reaction can be presented as C/T, serving as an intuitive “stress index” based on the anabolic-catabolic equilibrium.

Referring to FIG. 6, illustrated is a graphical representation 600 of a population sample for real-time, single-point hormonal stress analysis, in accordance with an embodiment of the present disclosure. As shown in the figure, the graphical representation demonstrates the statistical variation in the cortisol values throughout the day and shown how individual samples are deviating from the mean can indicate higher or lower than normal stress levels.

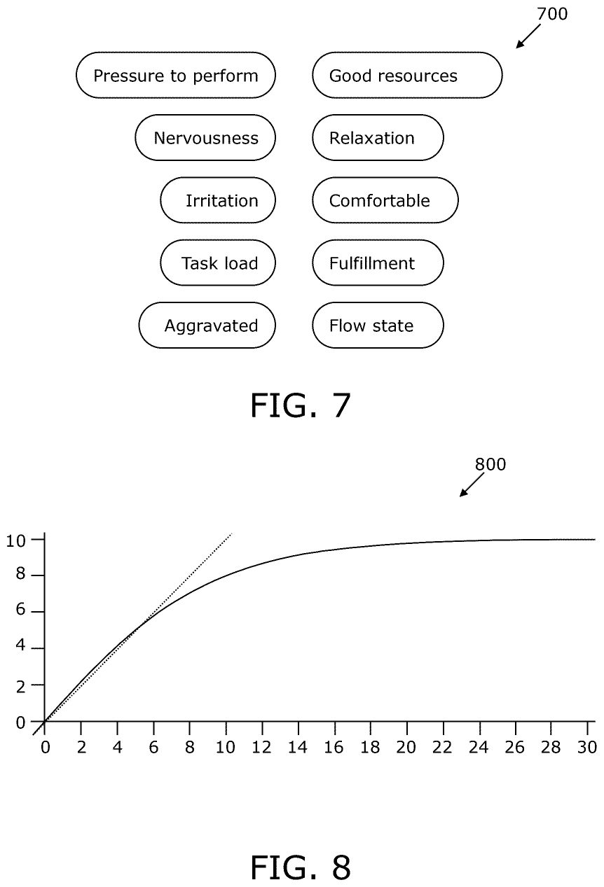

Referring to FIG. 7, illustrated is an exemplary implementation 700 of a set of predetermined tags, in accordance with an embodiment of the present disclosure. As shown in the figure, the set of predetermined tags is used to assess the mental stress. Five positive (recovery) tags and five negative (stress) tags are presented, such as “Pressure to perform,” “Nervousness,” “Relaxation,” and “Flow state.” These tags are selected by the user to gauge their mental load and resilience.

Referring to FIG. 8, illustrated is an exemplary behaviour 800 of a transformation function of a set of predetermined tags, in accordance with an embodiment of the present disclosure. As shown in the figure, the selected transformation function (a generalized logistic function) is used to convert the calculated stress value into an intuitively understandable scale of [0, 10]. The transformation function behaves nearly linearly in the lower range and taper offs as the transformation function approaches the upper limit.

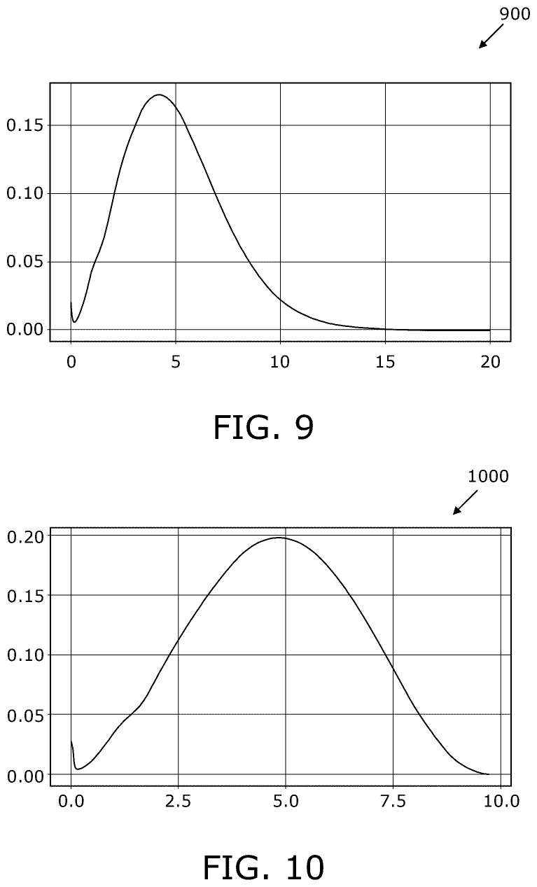

Referring to FIG. 9, illustrated is a graphical representation 900 depicting the distribution of user's adjusted mental load and flow score, in accordance with an embodiment of the present disclosure. As shown, the graphical representation presents a simulation of the distribution of the user's adjusted mental load and flow score. The X-axis denotes stress value of the user, and the Y-axis denotes the density. For example, if the user's adjusted mental load follows a normal distribution mental load˜N(5, 1.66) truncated to a range [0, 10] and the flow score follows a normal distribution flow score˜N(5, 1.66) truncated to a range [0, 10] similarly The user's adjusted mental load and the flow score both followed a truncated normal distribution. The resulting stress value is shown to be right-skewed.

Referring to FIG. 10, illustrated is a graphical representation 1000 depicting balancing of the user's adjusted mental load and flow score, in accordance with an embodiment of the present disclosure. As shown, the transformation function is applied to user's adjusted mental load and the flow score that followed the truncated path. After applying the transformation function, the final distribution of the stress amount resulting in a more balanced distribution centered around the midpoint of the scale.

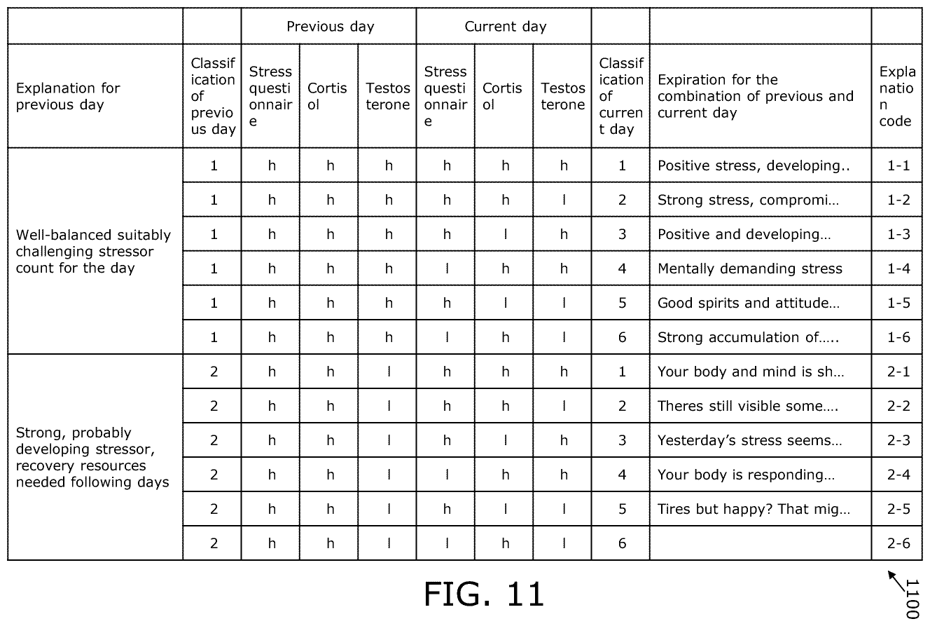

Referring to FIG. 11, illustrated is a table 1100 presenting the questions to assess exertion of a strength training session, in accordance with an embodiment of the present disclosure. The table 1100 depicts the questions that are used to assess the overall intensity (“exertion”) of a strength training session. The questions may include set amount, repetition amount, average per set effort level (i.e. are the sets performed into or close to voluntary maximum/until exhaustion). These questions form the basis for evaluating combined cardiovascular and neuromuscular strain.

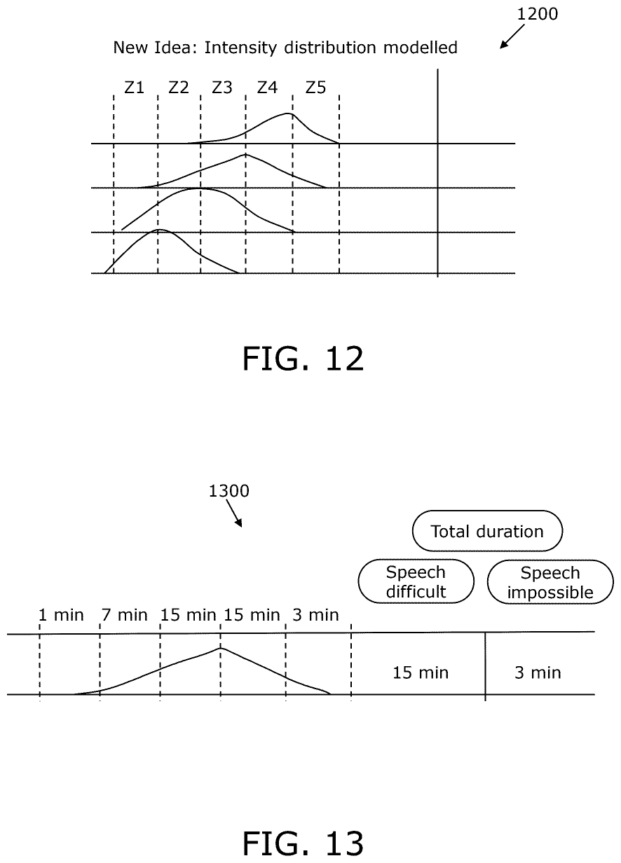

Referring to FIG. 12, illustrated is a graphical representation 1200 depicting modelling of the intensity (exertion) distribution, in accordance with an embodiment of the present disclosure. As shown, the graphical representation 1200 illustrates the principle of using a skewed distribution to model stress across different HR-zones (heart rate zones) during physical exertion. The more time spent in higher stress zones (zone 4 (speech difficult) and zone 5 (speech impossible)) (for example, described as the ability to complete a sentence or a word respectively), the more the distribution skews toward higher stress and work intensities. For an example, if combined the mental stress and the physical stress into a single combined stress parameter; select cortisol as the stress hormone and testosterone as the anabolic hormone, then classify the parameters each into only two categories, thus being “low” or “high”. There are 8 different combinations of combined stress, stress hormone and anabolic hormone for day 1. Furthermore, for the second day, which is called “reactive stress day”, may also be classified by the same 8 different combinations. What now emerges is that the user is able to classify the combinations of day 1 and day 2 together in 64 different ways (see “Current day” and “Explanation for the combination of previous and current day”).

Referring to FIG. 13, illustrated is a graphical representation 1300 depicting heart rate zone distribution modeling during a training session of a user, in accordance with an embodiment of the present disclosure. As shown, the modelling aimed at calculating training calories and related work volume metrics using known physiological parameters such as maximum heart rate (HR) and VO₂ max. When the total duration of a workout and the durations spent in heart rate zones Z5 and Z4 are known, the remaining time distribution across the lower heart rate zones can be modeled statistically in accordance with real HR recordings. A timeline with labeled durations is shown in the central portion of the figure. The timeline displays distinct durations spent in each heart rate zone, 1 minute in Zone 1 (Z1), 7 minutes in Zone 2 (Z2), 15 minutes in Zone 3 (Z3), 15 minutes in Zone 4 (Z4), 3 minutes in Zone 5 (Z5). The total workout duration is indicated as 41 minutes, with colored annotations identifying 15 minutes where speech is difficult and 3 minutes where speech is impossible, corresponding to higher intensity zones (Z4 and Z5). The statistical modeling is applied to skew the distribution in a manner that ensures the total workout duration is respected, particularly adjusting for the recorded durations in Z4 and Z5.

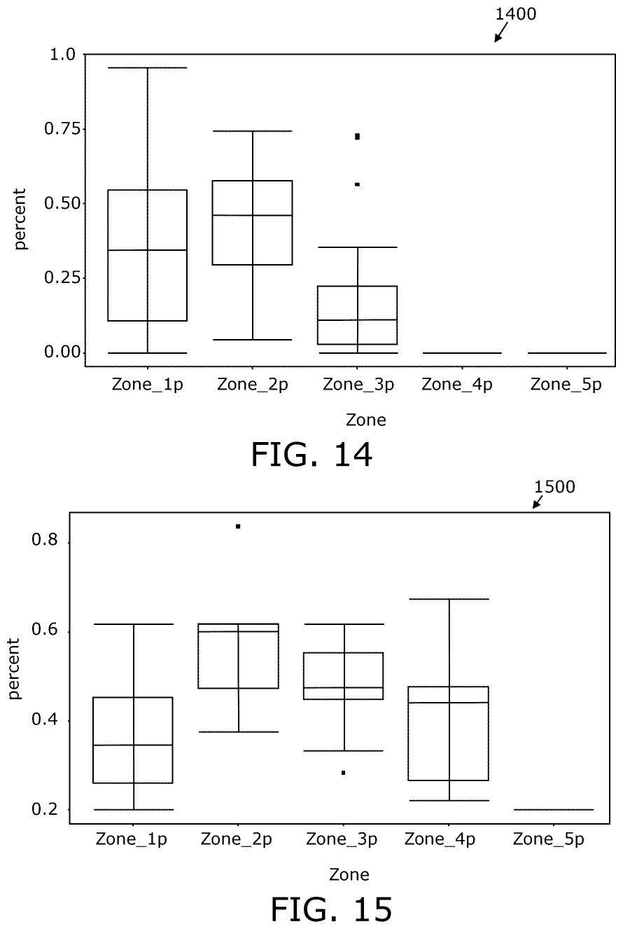

Referring to FIG. 14, illustrated is a boxplot representation 1400 depicting stress distribution across different heart rate training zones during exercise sessions of a user, in accordance with an embodiment of the present disclosure. In the figure, the X-axis represents the various heart rate zones, labelled as zone_1p, zone_2p, zone_3p, zone_4p, and zone_5p, while the Y-axis represents the percent of total training time spent in each respective zone. As shown, the user has exercised only in zones 1p-3p. Zone_2p is dominant, with some work in zone_3p. Zone_4p and zone_5p (Zones 4 and 5), corresponding to high- and very-high-intensity efforts, show minimal to no time allocation for the majority of the dataset, as indicated by near-zero median values.

Referring to FIG. 15, illustrated is a boxplot representation 1500 depicting statistical distribution of training session time percentages in different heart rate zones, in accordance with an embodiment of the present disclosure. In the figure, the X-axis represents the various heart rate zones, labelled as zone_1p, zone_2p, zone_3p, zone_4p, and zone_5p, while the Y-axis represents the percent of total training time allocated in each respective zone. As shown, the user has exercised with low intensity in zone_1p. Zone_2p is consistent and significant time spent at low-to-moderate intensity in this zone. In zone_3p indicates steady usage across users or sessions, with one minor outlier. Zone_4p and zone_5p (Zones 4 and 5), corresponding to high- and very-high-Intensity efforts, that the zone 4 is dominating over zone 5. It highlights that zones 2 to 4 comprise the majority of training time, offering insight into realistic heart rate distributions that can inform personalized training load estimations.

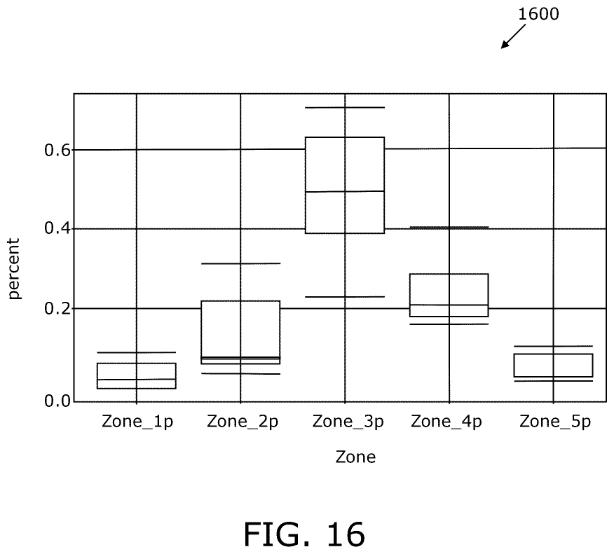

Referring to FIG. 16, illustrated is a boxplot representation 1600 depicting statistical distribution of training session time percentages in different heart rate zones, in accordance with another embodiment of the present disclosure. In the figure, the X-axis represents the various heart rate zones, labelled as zone_1p, zone_2p, zone_3p, zone_4p, and zone_5p, while the Y-axis represents the percent of total training time allocated in each respective zone. As shown, the zone 1 indicates that minimal time is spent in this zone across most sessions. Zone 2 indicates variability in its use, however only a small portion of training time is spent in this zone. As shown, the user has exercised with high volume in zone 3. Zone 4 indicates regular but varied engagement in high-intensity training zone and zone 5 indicates minimal and inconsistent time spent in maximum intensity zone. It highlights zone 3 as the primary training zone.

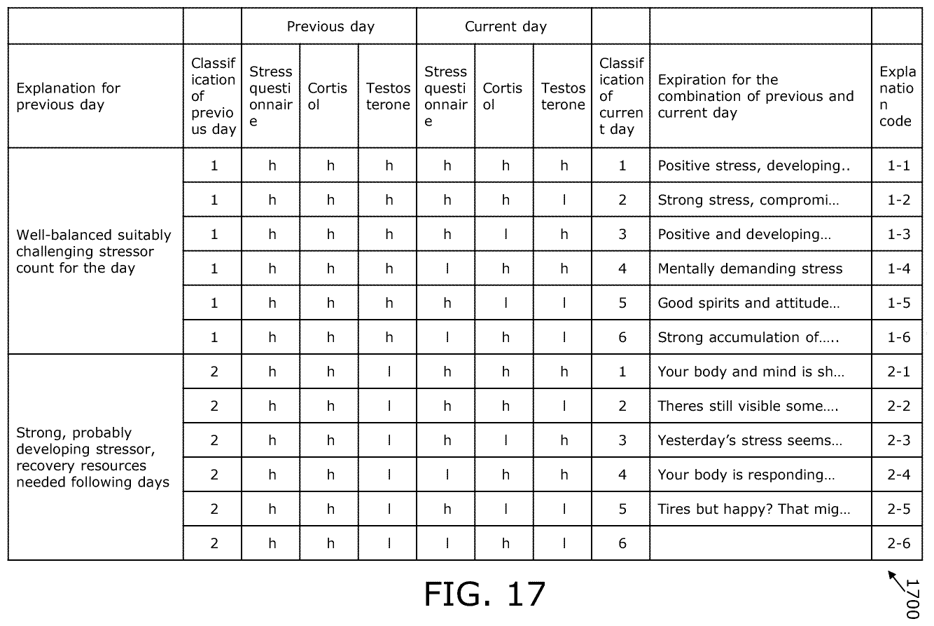

Referring to FIG. 17, illustrated is a tabular representation 1700 depicting analysis of intensity distribution across two consecutive days, in accordance with an embodiment of the present disclosure. As shown, the user's stress levels are classified across two consecutive days i.e., “previous day” and “current day” based on physiological and subjective stress indicators. The modelling facilitates the generation of personalized interpretations for an individual's stress progression and recovery needs. The tabular representation 1700 into three logical segments such as previous day indicators, current day indicators ad interpretive outputs. The previous day indicators comprises explanation for previous day, i.e. qualitative interpretation of the stress exposure (e.g., “well-balanced, suitably challenging stress count of the day”, “strong, probably developing stressor recovery resources needed following days”), classification for previous day i.e. numerical classification (1 or 2) to represent stress intensity or quality, self-reported data with values such as “h” (high), biomarker level, interpretated as high (“h”) or low (“l”). The current day indicators comprises stress questionnaire (self-reported stress level for the current day), cortisol and testosterone bio markers, numerical representation of stress status (1-8) for denoting severity to classy the current day. The interpretative outputs comprises an explanation for the combination of previous and current day i.e. textual insight (e.g., “Positive stress, developing capacity” or “Your body and mind is showing signs of strain”). A unique code (e.g., “1-2”, “2-5”) that maps each unique state transition to its associated explanation.

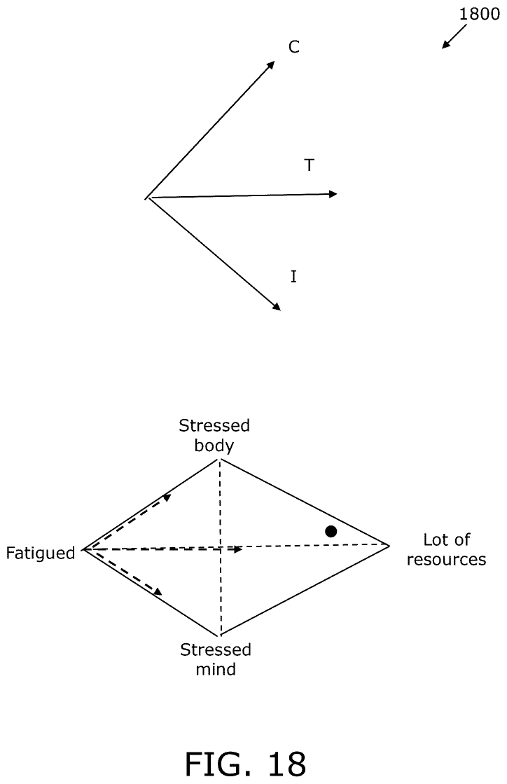

Referring to FIG. 18, illustrated is a graphical representation 1800 depicting stress and recovery of a user in a user interface, in accordance with an embodiment of the present disclosure. As shown, the graphical representation comprises (i) a vector space defined by the stress biomarkers and (ii) a diamond-shaped decision field mapping different user's stress state. The vector representation comprises vector C that is a stress hormone, vector T is anabolic hormone, vector I is combined mental+physical load. The origin serve as a baseline state. The Vector C is cortisol axis pointing upward, representing increased physiological stress levels. The vector T is testosterone axis extending horizontally, associated with energy, resilience and recovery capability. The vector I is inverse of testosterone or an indicator of immune or inflammatory state, pointing downward and reflecting decreasing adaptive capacity. The sum of these 3 vectors should be within the diamond-shaped area, which is roughly divisible into 4 different regions; on the left there is an area where subject is fatigued and depending on the horizontal location s/he may be more symptomatic on the mental or physiological parameters. Solid black dot indicates a specific state location (S=x, y), representing the user's current stress/resource status calculated from the biomarker vectors. Dashed arrows illustrate directional transitions (e.g., moving from fatigued to well-resourced, or from mind-stress to body-stress) based on evolving input conditions or recovery recommendations. It also has several implications for the interpretations that can be done (they are the outcome of the table), for example we may detect high mental resilience (high stress, high stress hormones but good spirits) or high physiological resilience (high stress hormone, high mental stress but high anabolic hormones). High physiological resilience is always present when the sum is on the right side, since anabolic hormone represents high capacity to overcome stressors. The right side is also vertically divided into mental or physical stress and provides the possibility to interpret if the anabolic capacity is able to overcome physiologically or mentally manifested stressors. Optionally, the diamond shaped plotting area may be twisted to form a regular quadrant shape as well as the starting point for the vectors may be located in the corner, in the middle of the field or on any side of the map.

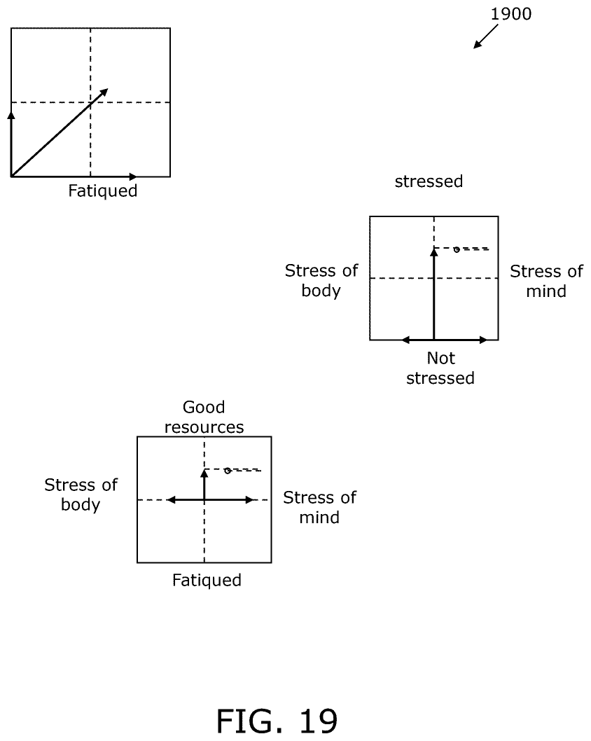

Referring to FIG. 19, illustrated is a graphical representation 1900 depicting a stress modelling approach of an exemplary scenario, in accordance with an embodiment of the present disclosure. As shown, the figure maps the stress state of an individual in a two-dimensional coordinate space where the X-axis represents cognitive or mental stress (T), and the Y-axis represents physical stress (C). The vector sum of C and T places a point in a quadrant defined by “Fatigued”, “Stressed Body”, “Stressed Mind”, and “Lots of Resources”. The graph conveys the combined state of an individual's physiological and psychological stress along with their overall energy/resource status. A point towards the right indicates resource sufficiency despite stress, while a point to the left indicates fatigue. Moreover, the arrows represent the individual contributions of body and mind stress toward a net state. The vector pointing towards the top right quadrant indicates increasing combined stress, while the dashed lines act as a reference for axis midpoints. This visual aids in representing stress evolution over time and guides visualization of movement toward or away from healthy states. The figure represents a dot that indicates the current state of an individual, lying in the top-right quadrant, signifying both mental and physical stress. This visual supports a dual analysis of stress against available recovery capacity or resilience, forming the basis for adaptive stress management strategies.

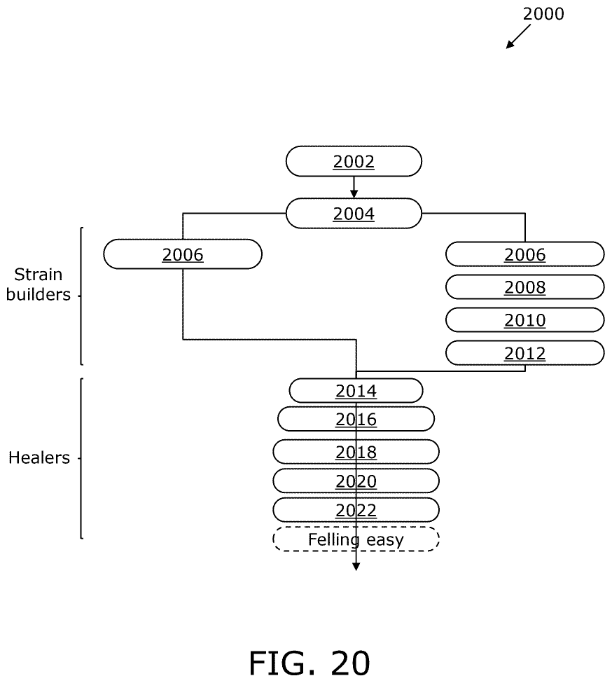

Referring to FIG. 20, illustrated is a flow chart 2000 depicting steps of a stress evaluation method for physical stress, in accordance with an embodiment of the present disclosure. At step 2002, session duration is assessed. At step 2004, physical stress is divided into two primary categories i.e. cardiovascular physical stress 2006 and strength-based stress, based on the nature of exercise. Step 2006 depicts high sets. Step 2008 depicts high repetitions. Step 2010 depicts maxing out (lifting to near failure). Step 2012 depicts big weights (heavy resistance) (for example, big weights 1-6 RM, moderate weights 6-12 RM, light weights 12+ RM). The aforementioned activities are categorized under the label “strain builders”, contribute to accumulated physical stress. To counteract the stress induced by the strain builders, the flow chart 2000 introduces a sequence of “Healers” i.e. strategies that aid in stress reduction and physical recovery. Step 2014 depicts rest day i.e. complete cessation of training to allow-body recovery. Step 2016 depicts recovery exercises i.e. low intensity movement to promote blood flow and reduce soreness. Step 2018 depicts treatment that may include physical therapy, massage and the like. Step 2020 depicts naps i.e. short sleep intervals. Step 2022 depicts excellent nutrition i.e. dietary strategies aimed at replenishing energy stores and supporting muscle repair. The aforementioned interventions lead to endpoint “Feeling easy”, which is enclosed in a dashed outline, indicating a desired state of reduced stress and readiness for future exertion.

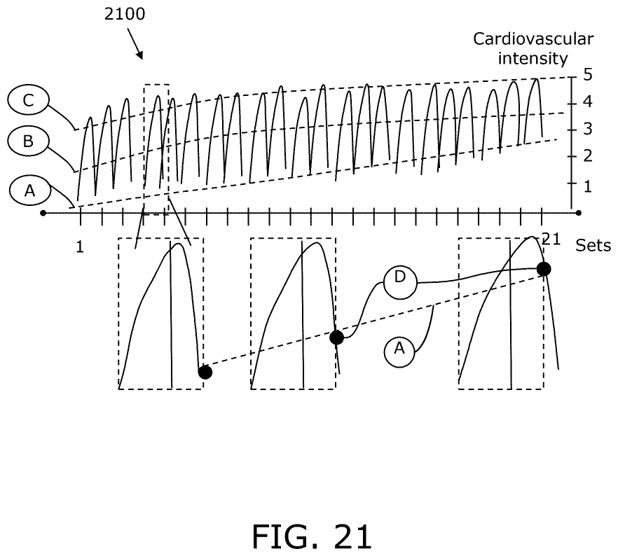

Referring to FIG. 21, illustrated is a graphical representation 2100 depicting fatigue and stress accumulation during a strength training session, in accordance with an embodiment of the present disclosure. As shown, total fatigue and stress of the strength training session is an integral of consecutive single-set fatigue curves, having an interaction via A_n and A_n−1, which are the function of E, F and B_n. The X-axis represents the time or the sequence of sets, while the Y-axis represents the level of accumulated fatigue or stress, which may be measured by physiological parameters such as heart rate (HR). Each curve in the figure corresponds to the fatigue profile of an individual set. As a set begins, fatigue accumulates rapidly, reaching a local maximum at the end of the set. Following the set, a recovery phase occurs during the inter-set rest period, during which fatigue partially dissipates. However, if the rest period is insufficient or the sets are performed in close succession, the baseline fatigue before each subsequent set increases, leading to a progressive accumulation of fatigue across the session. The figure highlights the interaction between consecutive sets, denoted as A_n and A_n−1, which are functions of variables such as exercise intensity (set and interest duration) (E), interest duration (F), and set-specific parameters (highest cardiovascular intensity) (B_n). Over time, as sets are repeated with short rest intervals or high intensity, the curves for Aₙ (lowest cardiovascular intensity of the interest) and Aₙ₋₁ (lowest cardiovascular intensity of the previous interest) converge, indicating that the user is unable to fully recover between sets. This results in a blurring of the setwise fatigue pattern, and the overall fatigue buildup becomes more continuous, as reflected in physiological markers like HR. After collecting information 1-5 at least partially, the model of the strength training session physical stress can be utilized. The model is based on the quite a uniform way of muscular effort during sets, which consists of repetitions. Physical work during the exercise set is accumulating fatigue repetition by repetition, leading into, for example, an increasing heart rate, which will reach a local maximum soon after the last repetition. After that, the fatigue will start to vanish, visible for example in decreasing HR. The fatigue washout will continue until the next set starts.

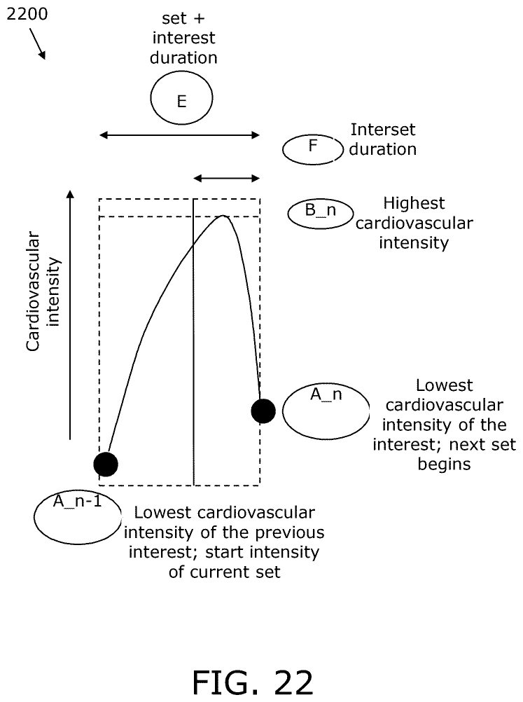

Referring to FIG. 22, illustrated is a graphical representation 2200 depicting fatigue and stress accumulation during a single exercise set, in accordance with an embodiment of the present disclosure. As shown, as the training continues the number of current set (n) increases, E stays the same, A_n increases, B_n increases. The increase in both A_n and B_n slows as the n increases and converging on the maximum cardiovascular intensity the user can achieve. Moreover, F increases if NO “high set”, “high rep”. conversely, “high set”, “high rep” decreases F. furthermore, the distance between B_n and A_n increases after “maxing out” elevates B_n. The “big weights” in only counted on neuromuscular strain.

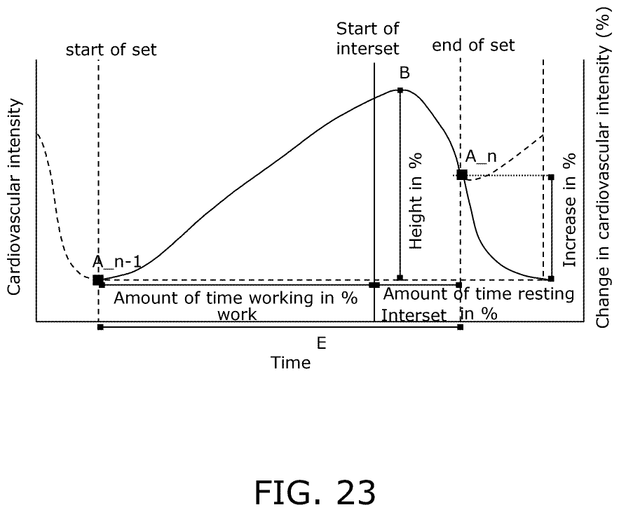

Referring to FIG. 23, illustrated is a graphical representation 2300 depicting fatigue and stress accumulation across multiple sets and repetitions, in accordance with an embodiment of the present disclosure. The figure integrates the features from FIG. 21 and FIG. 22. The figure shows a single-set fatigue curve, corresponding to an individual set within the session. The X-axis represents the time or the sequence of sets, while the Y-axis represents the cardiovascular intensity, which may be measured by physiological parameters such as heart rate (HR). The total fatigue and stress of the strength training session is mathematically described as the integral of consecutive single-set fatigue curves, with each curve interacting with the previous one. The curve for Aₙ (lowest cardiovascular intensity of the current set) denotes the end of a set and Aₙ₋₁ (lowest cardiovascular intensity of the previous interest) denoted the start of a set. The A_n and A_n−1 are functions of variables such as exercise intensity (set and interest duration) (E), interest duration (F), and set-specific parameters (highest cardiovascular intensity) (B_n) converge as the session continues, especially when sets are repeated with short rest intervals or high intensity, the curves for A_n and An−1 become increasingly similar, indicating that the user is unable to fully recover between sets. Eventually, the fatigue buildup (as measured, for example, by HR) loses its distinct setwise pattern and becomes a more continuous, sustained elevation.

1. A method for defining a stress level, the method comprising:
obtaining a first set of information related to mental stress from a user via a first questionary;
obtaining a second set of information related to physical stress from the user via a second questionary;
measuring a hormonal level at a first moment of time;
using the first and second sets of information and the measured hormonal level to derive a stress reaction over a pre-defined period of time;
validating the stress reaction using longitudinal stress reaction; and
defining the stress level by modelling stress level from at least one of:
the stress reaction from at least one of: the first and second questionaries;
the validated stress reaction.

2. The method of claim 1, wherein the mental stress comprises:
a set of mental load to be selected by the user on a scale of 0 to 10; and
a plurality of tags indicative of a user's perception of the mental load, wherein each tag has a score on a scale of 0 to 5, to be selected by the user.

3. The method of claim 2, wherein the plurality of tags comprises:
negative tags, associated with stress, selected from at least one of: good resources, relaxation, comfortable, fulfilment, flow state; and
positive tags, associated with recovery, selected from at least one of: pressure to perform, nervousness, irritation, task load, aggravated.

4. The method of claim 2, wherein the step of deriving the stress reaction further comprises:
scaling the mental load to produce an adjusted mental load, using a relation
$${Mental}_{adj} = ( \frac{Mental load - 1}{9} ) * 10 ;$$
computing a flow score from the selected tags, wherein the flow score is calculated by subtracting the sum of the tag scores from a predetermined baseline value using a relation
$$Flow score = 5 - ( sum of tag scores ) ;$$
weighting the adjusted mental load with the flow score to determine an initial stress level using a relation:
$$Initial Stress Value = {Mental}_{adj} * ( \frac{10 - Flow score}{5} ) ;$$
and
applying a transformation function to the initial stress level such that the transformed stress level lies within a normalized range using a relation
$$T ( X ) = ( - 1 + \frac{1 + 1}{1 + {e}^{- 0.22 * 10 * ( \frac{X}{10} )}} ) * 10.$$

5. The method of claim 4, wherein the transformation function enables transforming the stress level to be measured on a scale of 1 to 10.

6. The method of claim 4, wherein the transformation function is a generalized logistic function.

7. The method of claim 1, wherein the second questionary is associated with a set of physical stress parameters comprising at least one of:
a duration of performing physical task;
a duration of performing physical task when the user is not able to speak more than one word; and
a duration of performing physical task until exhaustion.

8. The method of claim 1, wherein the step of longitudinal stress reaction is based on the hormonal level.

9. The method of claim 1, wherein the step of measuring the hormonal level at the first moment of time uses dual markers for stress and anabolism, and wherein the dual markers are selected from:
a stress marker comprising cortisol (C); and
an anabolic marker comprising testosterone (T).

10. The method of claim 1, further comprising hormone-based stress ignition-consequence tracking based on the hormonal level over the predefined period of time, wherein the hormonal levels comprise: a stress marker, and an anabolic marker.

11. The method of claim 10, wherein
a hormone-based stress ignition is indicated by an absolute concentration of the stress marker, wherein the stress marker comprises cortisol; and
a magnitude of the hormone-based stress ignition is indicated by a change in equilibrium of concentrations of the stress marker and the anabolic marker, wherein the anabolic marker comprises testosterone (T).

12. The method of claim 10, wherein
a hormone-based stress consequence is indicated by a area under curve of the change in equilibrium of the concentrations of the stress marker and the anabolic marker; and
a recovery of resources and healing processes is indicated by a concentration of the anabolic marker.

13. The method of claim 12, wherein the concentration of the anabolic marker is indicative of a good stress or a bad stress, and wherein the bad stress is indicated by higher stress marker and good stress is indicated by a higher anabolic marker.

14. The method of claim 1, further comprising storing, in a database, responses to the obtained first and second questionaries and hormonal levels.

15. The method of claim 1, further comprising employing a deep neural network (DNN) algorithm trained on a population data set to model user-specific stress patterns and to refine the derivation of the stress level.

16. The method of claim 1, wherein the stress level is visually represented in any of: a tabular form, a graphical form.

17. A system for defining a stress level, the system comprising a processor configured to:
obtain, via a user interface communicably coupled to the processor, a first set of information related to mental stress from a user via a first questionary;
obtain, via a user interface, a second set of information related to physical stress from the user via a second questionary;
receive a hormonal level, measured using a sensor arrangement, at a first moment of time;
use the first and second sets of information and the measured hormonal level to derive a stress reaction over a pre-defined period of time;
validate the stress reaction using longitudinal stress reaction; and
define the stress level by modelling stress level from at least one of:
the stress reaction from at least one of: the first and second questionaries;
the validated stress reaction; and
display, on the user interface, the stress level as a visual representation.

18. The system of claim 17, further comprising a database configured to store responses to the obtained first and second questionaries and hormonal levels.

19. The system of claim 17, further comprising a deep neural network (DNN) algorithm trained on a population data set to model user-specific stress patterns and to refine the derivation of the stress level.

20. A computer program product comprising a non-transitory machine-readable data storage medium having stored thereon program instructions that, when executed by a processor, cause the processor to execute steps of the method of claim 1.
